(19) United States
(12) Patent Application Publication
(10) Pub. No.: US 2014/0363835 A1
(43) Pub. Date: Dec. 11, 2014

(54) COMPATIBLE SOLUTE ECTOINE AS WELL AS DERIVATIVES THEREOF FOR ENZYME STABILIZATION

(71) Applicant: Roche Diagnostics Operations, Inc., Indianapolis, IN (US)

(72) Inventors: Gabriele Chemnitius, Mannheim (DE); Otto Gaa, Worms (DE); Thomas Nagel, Mannheim (DE); Karl Recht, Buerstadt (DE)

(21) Appl. No.: 14/466,264

(22) Filed: Aug. 22, 2014

Related U.S. Application Data
(63) Continuation of application No. PCT/EP2013/054351, filed on Mar. 5, 2013.

(30) Foreign Application Priority Data
Mar. 6, 2012 (EP) 12158286.0

(51) Int. Cl. C12Q 1/32

(52) U.S. Cl. CPC C12Q 1/32; G01N 2333/902
USPC 435/14; 435/26

(57) ABSTRACT

Dry reagent compositions are disclosed for maintaining and preserving enzymatic activity. Also disclosed are test elements incorporating the same and methods of making and using the same for determining the presence or amount of an analyte in a body fluid sample. The dry reagent compositions can include components such as a dehydrogenase, a redox cofactor, an agent capable of eliciting at least one optical change in an optical property of an indicator reagent in the presence of redox equivalents, an indicator reagent, and at least one compatible solute being ectoine or a derivative thereof.

### CROSS-REFERENCE TO RELATED APPLICATIONS

This patent application is a continuation of Int'l Patent Application No. PCT/EP2013/054351; filed 5 Mar. 2013, which claims priority to and the benefit of EP Patent Application No. 12158286.0; filed 6 Mar. 2012.

### TECHNICAL FIELD

This disclosure relates generally to chemistry and medicine/medical diagnostics, and more particularly, it relates to stabilized dry reagent compositions and diagnostic test elements including the same for determining an analyte concentration in a body fluid sample.

### BACKGROUND

Diagnostic test elements usually are manufactured for use in near-patient applications. The elements therefore must be robust with respect to handling and storage. This applies, in particular, for the test chemistry of test elements. See, e.g., Hönes et al. (2008) Diabetes Technol. Ther. 10:S10-S26.

Many test elements are based on rather complex enzyme-based test chemistries. For example, some test elements include a carrier and a detection layer, where the detection layer contains enzymes. It is decisive for the proper function of the test elements that these enzymes remain biologically active during storage and upon subsequent use. Since calibration for an individual measurement usually is not possible, the test elements typically are calibrated batch-wise. The calibration information for a batch of test elements is stored and used for each test element of the batch regardless of individual differences in treatment and storage.

Pretreatments of the test elements and storage conditions can severely affect enzymatic activity. For example, heat treatment, either during manufacture or storage of the test elements, can denature the enzymes such that overall enzymatic activity of a test element is significantly reduced which, in turn, will result in inaccurate results when such a test element is used. Similarly, many enzymes are sensitive to oxidative processes, which also results in denaturation and irreversible enzyme inactivation. Likewise, many enzymes on test elements are present, at least during the time of storage, in a solvent-free environment that may promote such oxidative processes. Moreover, the detection layer may include additional components that can facilitate oxidative processes such as redox cofactors and other redox-relevant components and the like.

The problem of preserving enzymatic activities under such unfavorable conditions does not apply only to test elements. Rather, more generally, many enzyme preparations are provided and stored in essentially solvent-free form, such as freeze-dried preparations.

Various so-called compatible solutes (i.e., low molecular weight compounds of different chemical classes), such as sugars, polyols, free amino acids, amino acid derivatives, amines and sulphur analogs, sulfate esters, short peptides and cyclic 2,3-diphosphoglycerate, have been investigated for their preservative properties for proteins in solution and under dry conditions. See, e.g., Arakawa & Timasheff (1985) Biophys. J. 47:411-414; Lippert & Galinski (1992) Appl. Microbiol. Biotechnol. 37:61-65; Göller (1999) J. Mol. Catal. B-Enzym. 7:37-45; and Lentzen & Schwarz (2006) Appl. Microbiol. Biotechnol. 72:623-634; as well as WO Patent Application Publication No. 2007/002657 and US Patent Application Publication No. 2010/0255120.

Stabilization of glucose oxidase by ectoine and hydroxyectoine in test elements for electrochemically determining glucose in solution has been previously reported. See, e.g., WO Patent Application Publication No. 2007/097653; and Loose (2006) Proceedings of the 24th IASTED Int'l Multi-Conference Biomedical Engineering, 167-173 (Innsbruck, AT). Glucose oxidase, however, is known to be a rather stable enzyme with respect to oxidative stress and heat. On the other hand, hydroxyectoine was reported to be incapable of preventing protein aggregation in solution for lactate dehydrogenase. See, Andersson et al. (2000) Biotechnol. Appl. Biochem. 32:145-153.

Dehydrogenases in general and, in particular, glucose dehydrogenases are rather sensitive enzymes for oxidative stress and heat treatment. However, they are important diagnostic tools. Ectoine also has been reported as agent in cholesterol test elements including cholesterol dehydrogenase aiming for electrochemical detection of the enzymatic activity. See, e.g., WO Patent Application Publication Nos. 2007/132226 and 2006/067424.

Therefore, there is a need for preserving enzymatic activity of storage- and temperature-sensitive enzymes used for diagnostic applications especially a class of dehydrogenases such as glucose dehydrogenase.

### BRIEF SUMMARY

An inventive concept described herein is that ectoine and/or derivatives thereof can be used as a compatible solute to improve enzymatic activity of storage- and temperature-sensitive dehydrogenases that are used in diagnostic test elements. This inventive concept can be incorporated into exemplary compositions, devices and methods as described herein.

In one aspect, dry reagent compositions are provided for maintaining and preserving enzymatic activity that include (a) a dehydrogenase, (b) a redox cofactor, (c) an agent capable of eliciting at least one optical change in an optical property of an indicator reagent in the presence of redox equivalents, (d) an indicator reagent, and (e) at least one compatible solute being ectoine or a derivative thereof. The at least one compatible solute is present in amounts of at least about 3 (w/w) %, at least about 4 (w/w) %, at least about 5 (w/w) %, at least about 6 (w/w) %, at least about 7 (w/w) % or at least about 8 (w/w) %.

The dry reagent compositions are a solid composition under normal conditions (i.e., under room temperature and normal pressure).

In some instances, the dehydrogenase can be lactate dehydrogenase, glucose dehydrogenases, alcohol dehydrogenase, L-amino acid dehydrogenase, glycerin dehydrogenase, malate dehydrogenase, 3-hydroxybutyrate dehydrogenase or sorbitol dehydrogenase. In other instances, the dehydrogenase is a glucose dehydrogenase, quinoprotein glucose dehydrogenase (e.g., pyrrolo quinoline quinone (PQQ)-dependent glucose dehydrogenase), glucose-6-phospate dehydrogenase, nicotinamide adenine dinucleotide (NAD)-dependent glucose dehydrogenase, flavin adenine dinucleotide (FAD)-dependent glucose dehydrogenase or enzymatically active mutants thereof. In certain instances, the enzymatically active mutant is a glucose dehydrogenase having a mutation at least at amino acid position 96, 170 and/or 252.

In some instances, the redox cofactor can be pyrrolo quinoline quinone (PQQ), nicotinamide-adenine-dinucleotide (NAD) or a derivative thereof, or a flavine cofactor such as flavin-adenine-dinucleotide (FAD) or flavine mononucleotide (FMN). In other instances, the redox cofactor can be carba-NAD.

In some instances, the agent capable of eliciting at least one optical change in an optical property of an indicator reagent in the presence of redox equivalents can be phenazine such as phenazinethosulfate, phenazinmethosulfate, 1-(3-carboxypropoxy)-5-ethylphenaziniumtrifluoromethansulfonate or 1-methoxyphenazinmethosulfate. In other instances, the agent can be a chinone such as phenanthrenchinone, phenanthrolinchinone or benzo[h]-chinolinchinone. In other instances, the agent can be a nitrosoaniline such as [(4-nitrosophenyl)imino]dimethanol-hydrochloride. In other instances, the agent can be a diaphorase such as a lipoamide deydrogenase, a NADH dehydrogenase or an enzymatically active mutant thereof.

In some instances, the indicator reagent can be a heteropoly acid such as 2,18-phosphoromolybdenic acid, a chinone such as resazurine, dichlorophenolindophenole or tetrazolinum salts. In other instances, the indicator reagent can be a fluorophore such as flavine nucleotides and nicotine-adenine-dinucleotides.

In some instances, the ectoine derivative can be hydroxyectoine, homoectoine, a hydroxyectoine ester, a hydroxyectoine ether, a sulfonyl derivative of ectoine, an esterified sulfonyl derivative of ectoine or an amide of a sulfonyl derivative of ectoine.

The dry reagent compositions also can include at least one stabilizer, detergent, swelling agent, film-forming agent and/or solid particle.

In another aspect, diagnostic test elements are provided for determining an analyte amount (or concentration) or presence from a body fluid sample, where the diagnostic test elements include a dry reagent composition as described herein.

In view of the forgoing, methods are provided for manufacturing the diagnostic test elements as described herein.

In another aspect, methods are provided for using compositions including at least one compatible solute as described herein for maintaining/preserving (i.e., attenuating a decrease in) enzymatic activity of at least one enzyme in a dry reagent composition.

In another aspect, methods are provided for determining an amount (or concentration) or presence of an analyte in a body fluid sample with a test element as described herein.

These and other advantages, effects, features and objects of the inventive concept will become better understood from the description that follows. In the description, reference is made to the accompanying drawings, which form a part hereof and in which there is shown by way of illustration, not limitation, embodiments of the inventive concept.

### BRIEF DESCRIPTION OF THE DRAWINGS

The advantages, effects, features and objects other than those set forth above will become more readily apparent when consideration is given to the detailed description below. Such detailed description makes reference to the following drawings, wherein:

FIG. 1 shows average values for activity and are indicated compared to the amount of stabilizer (i.e., compatible solute). The activity determined in test elements without stabilizer has been subtracted.

FIG. 2 shows a graphic view of the stabilizing effect of ectoine and hydroxyectoine on glucose dehydrogenase and diaphorase.

FIGS. 3A-C show graphic views of storage temperature compared to the measured glucose level as an indicator for the enzymatic activity. A) without stabilizer; B) ectoine, 1 g/100 g composition; and C) hydroxyectoine, 1 g/100 g composition.

FIGS. 4A-D show influences of different buffers used in the coating compositions. A) PO₄-buffer pH 6.8, without stabilizer, after 45 days, 45° C. to 4° C.; B) PO₄-buffer pH 6.8, 2 g ectoine/100 g RF, after 45 days, 45° C. to 4° C.; C) HEPES pH 7.1, without stabilizer, after 45 days, 45° C. to 4° C.; and D) HEPES pH 7.1, 2 g ectoine/100 g RF, after 45 days, 45° C. to 4° C.

FIG. 5 shows that a stabilizing effect of ectoine on Gluc-DOR and Gluc-DOR 31 mutant is observable after 3 weeks at 45° C.

Corresponding reference characters indicate corresponding parts throughout the several views of the drawings.

While the inventive concept is susceptible to various modifications and alternative forms, exemplary embodiments thereof are shown by way of example in the drawings and are herein described in detail. It should be understood, however, that the description of exemplary embodiments that follows is not intended to limit the inventive concept to the particular forms disclosed, but on the contrary, the intention is to cover all advantages, effects, features and objects falling within the spirit and scope thereof as defined by the embodiments described herein and the claims below. Reference should therefore be made to the embodiments described herein and claims below for interpreting the scope of the inventive concept. As such, it should be noted that the embodiments described herein may have advantages, effects, features and objects useful in solving other problems.

### DESCRIPTION OF EXEMPLARY EMBODIMENTS

The compositions, devices and methods now will be described more fully hereinafter with reference to the accompanying drawings, in which some, but not all embodiments of the inventive concept are shown. Indeed, the compositions, devices and methods may be embodied in many different forms and should not be construed as limited to the embodiments set forth herein; rather, these embodiments are provided so that this disclosure will satisfy applicable legal requirements.

Likewise, many modifications and other embodiments of the compositions, devices and methods described herein will come to mind to one of skill in the art to which the disclosure pertains having the benefit of the teachings presented in the foregoing descriptions and the associated drawings. Therefore, it is to be understood that the compositions, devices and methods are not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the disclosure pertains. Although any methods and materials similar to or equivalent to those described herein can be used in the practice or testing of the compositions, devices and methods, the preferred materials and methods are described herein.

Moreover, reference to an element by the indefinite article “a” or “an” does not exclude the possibility that more than one element is present, unless the context clearly requires that there be one and only one element. The indefinite article “a” or “an” thus usually means “at least one.” Likewise, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. For example, the expressions “A has B,” “A comprises B” and “A includes B” may both refer to a situation in which, besides B, no other element is present in A (i.e., a situation in which A solely and exclusively consists of B) or to a situation in which, besides B, one or more further elements are present in A, such as element C, elements C and D, or even further elements.

Overview

Techniques for removing solvent from a dry reagent composition, such as heat treatment, are known to affect enzymatic activity of sensitive enzymes such as dehydrogenases. Moreover, under dry conditions, enzymes such as dehydrogenases become more sensitive oxidative processes and accompanying enzyme denaturation. Accordingly, the manufacture of complex, dry compositions for enzymatic detection assays as well as the storage thereof often is accompanied by increasing inactivation of the enzymes by denaturation, aggregation or other processes. Upon reconstitution of the enzymes in a solvent-containing surrounding, a reduced enzymatic activity is observed. It has been found in the studies supporting the present disclosure that the reduction of enzymatic activity that can occur during manufacture and/or storage of the complex dry compositions can be significantly prevented by adding at least one compatible solute as specified elsewhere herein in detail. The finding is surprising since ectoine and derivatives thereof have been reported to be insufficient to prevent aggregation of, for example, lactate dehydrogenase, which also results in a reduction of enzymatic activity (Andersson et al. (2000), supra). Moreover, it is also surprising that the preservative effect occurs even under the redox sensitive conditions present in the rather complex, solvent-free composition. Interestingly, and also surprisingly, a preservative effect of ectoine was not observed in solution for the investigated dehydrogenases. In particular, the enzymatic activity present in a dry composition having the components as described herein except for the at least one compatible solute has been found to decrease in the dry state and during storage under dry conditions and temperatures above 4° C. In particular, it has been found that only about 50% of the enzymatic activity of a glucose dehydrogenase was present after 3 weeks storage at about 45° C. and only about 55% of the enzymatic activity of a diaphorase was maintained at these conditions. However, significantly higher enzymatic activities could be maintained when at least one compatible solute being ectoine or a derivative thereof was present in the dry reagent composition. A further advantage of the dry reagent compositions described herein is that the compatible solute applied in the composition does not interfere with optical detection systems. In particular, it does not interfere with the optical signals generated and also does not impair the stability or function of the indicator reagent, the redox cofactor or the agent capable of eliciting the at least one optical change. Moreover, the enzymatic conversion and conversion rates are not impaired by the compatible solute.

Dry Reagent Compositions

Compositions of the inventive concept include dry reagent compositions having the following components:
(a) a dehydrogenase;
(b) a redox cofactor;
(c) an agent capable of eliciting at least one change in an optical property of an indicator reagent in the presence of redox equivalents;
(d) an indicator reagent; and
(e) at least one compatible solute being ectoine or a derivative thereof.

Briefly, dry reagent compositions can be provided by dissolving these components first in a solvent or solvent mixture and subsequently removing the solvent or mixture of solvents by a suitable treatment as described in further detail below.

As used herein, “dry” means that the reagent composition is essentially free of a solvent or a mixture of solvents. As used herein, “essentially free” means that at least about 85%, at least about 90%, at least about 91%, at least about 92%, at least about 93%, at least about 94%, at least about 95%, at least about 96%, at least about 97% or even at least 98% of the solvent or solvent mixture that was originally present in a solution of the reagent composition is removed from the composition. Accordingly, it is contemplated that the solvent or solvent mixture is present in the dry reagent composition in an amount of up to about 15%, up to about 10%, up to about 9%, up to about 8%, up to about 7%, up to about 6%, up to about 5%, up to about 4%, up to about 3% or up to about 2%. The aforementioned percentage values and the other percentage values referred to herein refer to percent by weight (w/w).

As used herein, “about” means within a statistically meaningful range of a value or values such as, for example, a stated concentration, length, molecular weight, percentage, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

As noted above, one component of dry reagent compositions as described herein is a dehydrogenase. As used herein, “dehydrogenase” means a polypeptide that is capable of catalyzing an oxidation of a substrate by transferring hydrides (H⁻) as redox equivalents to an acceptor molecule, such as a redox cofactor as referred to herein elsewhere. Dehydrogenases can depend on a redox cofactor (also referred to as co-enzyme). Examples of dehydrogenases include, but are not limited to, lactate dehydrogenase (EC number 1.1.1.27 or 1.1.1.28), glucose dehydrogenases (see below), alcohol dehydrogenase (EC number 1.1.1.1 or 1.1.1.2), L-amino acid dehydrogenase (EC number 1.4.1.5), glycerin dehydrogenase (EC number 1.1.1.6), malate dehydrogenase (EC number 1.1.1.37), 3-hydroxybutyrate dehydrogenase (EC number 1.1.1.30) and sorbitol dehydrogenase (EC number 1.1.1.14).

In some instances, the dehydrogenase is a glucose dehydrogenase. Examples of glucose dehydrogenases include, but are not limited to, glucose dehydrogenase (EC number 1.1.1.47), quinoprotein glucose dehydrogenase (EC number 1.1.5.2), in particular, PQQ-dependent glucose dehydrogenase (EC number 1.1.5.2), glucose-6-phospate dehydrogenase (EC number 1.1.1.49), NAD-dependent glucose dehydrogenase (EC number 1.1.1.119) and FAD-dependent glucose dehydrogenase (EC number 1.1.99.10) and enzymatically active mutants thereof. In certain instances, the glucose dehydrogenase is a glucose dehydrogenase (E.C. 1.1.1.47) mutant disclosed in WO Patent Application Publication No. 2011/020856 having a mutation at least at amino acid position 96, 170 and/or 252. Examples of specific mutations at theses amino acid positions include, but are not limited to, substitutions of Glu96Gly, Glu170Arg or Lys and/or Lys252Leu.

The structure and properties of members of these enzyme families are well known in the art. See, Olsthoorn & Duine (1998) Biochem. 37:13854-13861; Pauly et al. (1976) Hoppe Seyler's Z Physiol. Chem. 356:1613-1623; and Tsujimura et al. (2006) Biosci. Biotechnol. Biochem. 70:654-659. Enzymatically active mutants of the aforementioned enzymes can be obtained by substituting, adding or deleting one or more amino acids from the amino acid sequences reported for the aforementioned wild type enzymes. Specific mutants are the mutants of the PQQ-dependent glucose dehydrogenase having an improved substrate specificity compared to the wild type counterparts as disclosed in U.S. Pat. Nos. 7,132,270 and 7,547,535. Further mutants are those disclosed in Baik et al. (2005) Appl. Environ. Microbiol. 71:3285-3293, Vasquez-Figuera et al. (2007) ChemBioChem 8:2295-2301, and WO Patent Application Publication No. 2005/045016.

Another component of the dry reagent compositions is a redox cofactor. As used herein, “redox cofactor” means a molecule that can serve as an acceptor for enzymatically transferred redox equivalents and, in particular, hydride (H—). As used herein, “redox equivalents” means hydrides (H⁻) that are transferred from a substrate of the dehydrogenase to the redox cofactor or electrons transferred to the indicator reagent from the redox cofactor.

The redox cofactor to be included in the dry reagent composition can vary depending upon the properties of the dehydrogenase used therein. Examples of redox cofactors include, but are not limited to, such as PQQ, NAD or a derivative thereof, or a flavine cofactor, such as FAD or FMN. Thus, for example, PQQ can be combined in a dry reagent composition with a PQQ-dependent glucose dehydrogenase, NAD can be combined in a dry reagent composition with a NAD-dependent glucose dehydrogenase, and FAD can be combined in a dry reagent composition with a FAD-dependent glucose dehydrogenase. In some instances, the redox cofactor can be a derivative of PQQ, NAD or FAD. Examples of NAD derivatives are disclosed in WO Patent Application Publication No. 2007/012494. In other instances, the redox cofactor can be carba-NAD as disclosed in WO Patent Application Publication No. 2007/012494.

Another component of the dry reagent compositions is an agent capable of eliciting at least one change in an optical property of an indicator reagent in the presence of redox equivalents. As used herein, “agent capable of eliciting a change in at least one optical property of an indicator reagent in the presence of redox equivalents” means a molecule, which in the presence of the redox equivalents, is capable of inducing a change in at least one optical property in an indicator reagent. The agent also may elicit a change in more than one optical properties of the indicator reagent, which may be subsequently detected. As used herein, an “optical property” means a property of the indicator reagent that can be optically detected, such as light absorption or emission, remission, refraction or polarization and properties associated therewith.

It is contemplated that such a change of at least one optical property as used herein encompasses the detection of the presence of a property that was not detectable before, the detection of the absence of a property that has been detected before and the detection of quantitative changes of a property (i.e., the detection of the change of the signal strength that correlates to the extent of the change of the at least optical property). Examples of optical properties include, but are not limited to, color, fluorescence, luminescence and refractometry. The optical properties that are to be changed by the agent thus depend on the type of indicator reagent. Depending upon the desired optical property to be detected and the agent to be used in the dry reagent composition, one of skill in the art is in a position to select without further ado a suitable indicator reagent, in particular among those referred to herein elsewhere.

An agent as referred to above can transfer directly or indirectly (i.e., via a further mediator) redox equivalents from the redox cofactor to the indicator reagent. As a consequence of the transfer of the redox equivalents, the indicator reagent will be modified such that a change in at least one optical property occurs. For example, a colorless or non-fluorescing indicator reagent in an oxidized state may be converted into a colored or fluorescent indicator reagent by the transfer of redox equivalents mediated by the agent in a reduced state.

An example of an agent capable of eliciting at least one change in an optical property of an indicator reagent in the presence of redox equivalents includes, but is not limited to, phenazine, especially phenazinethosulfate, phenazinmethosulfate, 1-(3-carboxypropoxy)-5-ethylphenaziniumtrifluoromethansulfonate or 1-methoxyphenazinmethosulfate. Phenazines can be applied for eliciting a change in at least one optical property of an indicator reagent. Details for the detection and on how such phenazines are to be applied can be found in EP 0 654 079 A1.

Another example of such an agent is chinone, especially phenanthrenchinone, phenanthrolinchinone or benzo[h]-chinolinchinone.

Another example of such an agent is nitrosoaniline, especially [(4-nitrosophenyl)imino]dimethanol-hydrochloride.

In some instances, the agent can be an enzyme capable of catalyzing the transfer of redox equivalents from the redox cofactor to the indicator reagent. An example of such an enzyme includes diaphorase (EC number 1.6.99.2), such as a lipoamide deydrogenase, a NADH dehydrogenase or an enzymatically active mutant thereof. Diaphorases can be obtained from pig heart, Clostridium kluyverii or Bacillus stearothermophilus. The structure of these enzymes is well-known in the art and described in, for example, DE Patent No. 2 061 984. Likewise, enzymatically active mutants can be used as described elsewhere herein. Additional diaphorases that can be used in the dry reagent composition are those having improved thermostability and catalytic properties as disclosed in US Patent Application Publication No. 2007/196899.

The transfer of the redox equivalents may be direct in that the redox equivalents are transferred by the agent to the indicator reagent or may be indirect. In the latter case, the redox equivalents are transferred from the agent to an intermediate mediator, which subsequently transfers the redox equivalents to the indicator reagent. It is contemplated that more than one mediator can be used. For example, the agent may transfer the redox equivalents to a first mediator which subsequently transfers the redox equivalents to a second mediator, and the second mediator then transfers the redox equivalents to the indicator reagent. In such a mediator cascade, more than two mediators can be used. An advantage of using one or more mediators for the transfer of the redox equivalents to the indicator reagent is that the timing of the optical detection can be improved. Examples of mediators include, but are not limited to, potassium ferricyanide, quinone derivatives, Nile blue (CAS no.: 3625-57-8), Meldola's blue (CAS no.: 7057-57-0), osmium complexes as disclosed in EP Patent No. 1 457 572 and transition metal complexes such as ruthenium hexamine chloride.

Another component of the dry reagent compositions is an indicator reagent. As used herein “indicator reagent” means a molecule or molecular entity that as a consequence of the transfer of redox equivalents will be modified so that a change in at least one optical property occurs. Examples of indicator reagents include, but are not limited to, heteropoly acids, preferably, 2,18-phosphoromolybdenic acid, chinones such as resazurine, dichlorophenolindophenole and/or tetrazolinum salts, especially the commercially available WST-3, WST-4 and WST-5 salts (Dojindo, Inc. US). These indicator reagents are reduced upon transfer of the redox equivalents, and the reduction is accompanied by a change in at least one optical property and, in particular, the color.

Other indicator reagents include fluorophores, the fluorescence of which is changed upon transfer of redox equivalents. Examples of fluorophores include, but are not limited to, flavine nucleotides and nicotine-adenine-dinucleotides referred to herein also in the context of the redox cofactors.

If a redox cofactor such as carba-NAD or NAD is applied in the dry reagent composition as an indicator reagent, the components (b), (c) and (d) may all be represented by the same molecule (i.e., the carba-NAD or NAD). Accordingly, components (b), (c) and (d) may be represented in the dry reagent composition by the same chemical entity. Moreover, a modified nitrosoaniline as disclosed in EP Patent Nos. 0 620 283 or 0 831 327 can be used as component (c) and component (d). Thus, components (c) and (d) may be represented in the dry reagent composition by the same chemical entity.

Another component of the dry reagent compositions is at least one compatible solute being ectoine or a derivative thereof. As used herein, “at least one compatible solute” means that two or more compatible solutes may be used together in the dry reagent compositions.

The at least one compatible solute reduces a decrease of the enzymatic activity of at least one enzyme in the reagent composition under dry conditions. The decrease of the enzymatic activity is prevented or at least significantly reduced in the dry reagent composition during manufacture and/or storage at room temperature or even higher temperatures as referred to above by the at least one compatible solute when compared to a control composition without the at least one compatible solute such that at least about 60%, at least about 70%, at least about 80% or at least about 90% of the enzymatic activity of one or both enzymes is maintained.

Ectoine (CAS number: 96702-03-3) is a well-known organic compound that naturally occurs in bacteria having the formula C6H10N2O2. It also is known as (S)-2-methyl-3,4,5,6-tetrahydropyrimidine-4-carboxylic acid. Ectoine is obtainable from various bacteria, in particular, of the genera Halomonadaceae or Firmicutes, by techniques well known in the art. See, e.g., WO Patent Application Publication No. 1994/15923).

As such, ectoine and one or more derivatives thereof can be included in the dry reagent compositions as described herein. As used herein, “derivative of ectoine” means a structurally related organic molecule capable of preventing a reduction of enzymatic activity of at least one enzyme as referred to herein in solution as well as in the dry state. The reduction of the enzymatic activity is prevented in a similar or the same manner and/or to a similar or the same extent as found for ectoine.

The derivative of ectoine, if present in a dry reagent composition, should prevent the reduction of enzymatic activity that occurs during manufacture and/or storage, in particular storage at room temperature or even higher temperatures such as, for example, about 45° C. or about 50° C., or even temperatures up to about 60° C., to a statistically significant extent. Thus, the derivative, if present in the dry reagent composition, should maintain at least about 60%, at least about 70%, at least about 80% or at least about 90% of the enzymatic activity of at least one enzyme in the dry reagent composition when compared to the enzymatic activity found in a control composition without the ectoine derivative. Examples of ectoine derivatives include, but are not limited to, hydroxyectoine, homoectoine, a hydroxyectoine ester, a hydroxyectoine ether, a sulfonyl derivative of ectoine, an esterified sulfonyl derivative of ectoine and an amide of a sulfonyl derivative of ectoine. Like ectoine, hydroxyectoine is obtainable from various bacteria, in particular, of the genera Halomonadaceae or Firmicutes, by techniques well known in the art. See, e.g., WO Patent Application Publication No. 1994/15923). The other ectoine derivatives can be obtained by, for example, chemical derivatizing hydroxyectoine in vitro. In some instances, a combination of derivatives of ectoine, such as hydroxyectoine and homoectoine, may also be used in the dry reagent composition.

The at least one compatible solute is present in amounts of at least about 3 (w/w) %, at least about 4 (w/w) %, at least about 5 (w/w) %, at least about 6 (w/w) %, at least about 7 (w/w) % or at least about 8 (w/w) %.

The prevention of the reduction of the enzymatic activity under dry conditions can be determined as described elsewhere herein.

In addition to the components described above, the dry reagent compositions can include at least one stabilizer, detergent, swelling agent, film-forming agent and/or solid particle. Suitable stabilizers, detergents, swelling agents, film forming agents, oxidizing agents, and/or solid particles that can be used in the dry reagent compositions are known to one of skill in the art.

An example of the at least one stabilizer includes polyvinylpyrrolidone, especially PVP K25.

Examples of the at least one detergent include, but are not limited to, sodium-N-methyl-N-oleoyltaurat, N-octanoyl-N-methyl-glucamid, Mega 8 (N-methyl-N-octanoylglucamide), dioctylsodium sulfosuccinate (DONS), Rhodapex® (especially CO-433 or CO-436).

Examples of the at least one swelling agent include, but are not limited to, methyl vinyl ether maleic acid anhydride copolymer, xanthan gum and methyl vinyl ether maleic acid copolymer.

Examples of the at least one film-forming agent include, but are not limited to, polyvinylpropionate dispersions, polyvinyl esters, polyvinyl acetates, polyacrylic esters, polymethacrylic acid, polyvinyl amides, polyamides, polystyrene and mixed polymerizates such as butadiene, styrene or maleic acid ester.

Examples of the at least one solid particle include, but are not limited to, silica particles such as silicon dioxide, sodium silicates or aluminium silicates, diatomaceous earth, metal oxides such as titan oxide and/or aluminium oxide, synthetic oxide materials such as nanoparticles of oxide materials such as nanoparticles of silicon dioxide, aluminium oxide, or titan oxide, Kaolin, powder glass, amorphous silica, calcium sulfate and barium sulfate.

In some instances, the dry reagent compositions include the components listed in Table 1 in the Examples below.

Devices

Devices of the inventive concept can include diagnostic test elements such as biosensors that include the dry reagent compositions as described herein.

Details on test elements and the manufacture thereof can be found in EP Patent No. 0 821 234 and also are described in additional detail below. Additional manufacturing details for test elements can be found in EP Patent Nos. 1 035 919 and 1 035 920.

In general, diagnostic test elements typically have a layered structure that is built upon a carrier. As used herein, “carrier” means a solid support that is used as a substrate onto which the dry reagent composition can be applied or immobilized. It is contemplated that the composition can be spatially arranged on the carrier. The carrier must be arranged in a manner to permit detecting of a change of the at least one optical property of the indicator reagent (i.e., it preferably does not comprise components or a spatial arrangement that would interfere with detecting the at least one optical property).

An example of a carrier includes vials containing the dry reagent composition (e.g., vials arranged in a well-plate format). Other assays may apply optical waveguides or semiconductor plates. Another example of a carrier includes test strips having one or more layers forming a solid carrier.

In addition to the carrier, diagnostic test elements include a test field containing a dry reagent composition as described herein, where the test field has a sample application side onto which a body fluid sample can be applied and a detection side that allows for detecting a change in an optical property when the analyte reacts with the dry reagent composition. It is contemplated that when the body fluid sample contains cells, such as erythrocytes present in blood samples, such cells do not reach the detection side.

Specifically, the test field includes a transparent foil onto which a first film layer and a second film layer are applied resting on top of one another in this order. It is important that the first layer located on the transparent foil scatters light considerably less than the overlying second layer. A non-coated side of the transparent foil is referred to as the detection side, and the side of the second layer that is opposite to the side with which the second layer rests on the first layer is referred to as the sample application side.

The film layers of the diagnostic test element can be produced from dispersions or emulsions of polymeric film formers. Dispersion film formers contain microscopic polymer particles that are insoluble in the carrier liquid (usually water) and are finely dispersed therein. If the carrier liquid is removed by evaporation during film formation, the particles come closer and finely touch one another. Large forces can occur in this process, and the gain in surface energy that accompanies the film formation results in the particles growing into a substantially closed film layer. Alternatively, it is possible to use an emulsion of the film former in which this is dissolved in a solvent. The dissolved polymer is emulsified in a carrier liquid that is immiscible with the solvent. Examples of polymers include, but are not limited to, polyvinyl esters, polyvinyl acetates, polyacrylic esters, polymethacrylic acid, polyvinyl amides, polyamides and polystyrene. In addition to homopolymers, mixed polymerizates are suitable, such as of butadiene, styrene or maleic acid ester.

The two film layers can be located on a transparent foil in the test field of the carrier. For this, the plastic foils come into consideration, which are impermeable to liquid. Polycarbonate foil has proven to be particularly suitable.

The two film layers can be produced from coating compounds that contain the same polymeric film formers, or they can be produced from coating compounds that contain different polymeric film formers.

Whereas the first layer contains a swelling agent and optionally a weakly light scattering filler, the second layer requires a swelling agent and in any case at least one pigment that scatters light strongly. In addition, the second layer can contain non-porous fillers as well as porous fillers.

By adding a swelling agent that swells well (i.e., a substance that increases its volume when it takes up water), one does not only obtain layers that can be penetrated relatively rapidly by sample liquid but have good cell (e.g., erythrocyte and additionally also blood pigment) separation properties despite this opening effect of the swelling agent. The swelling properties, however, should be so good that for a test in which the change of the at least one optical property is mainly dependent on the penetration of the sample liquid through the layer, the change of the optical property is measurable after a maximum of one minute. Examples of swelling agents include, but are not limited to, methyl vinyl ether maleic acid anhydride copolymer, xanthan gum and methyl vinyl ether maleic acid copolymer.

In some instances, the amount of the strongly light-scattering pigment in the second layer is at least about 25% by weight relative to the dry ready-to-use double layer of the test field. Since the weakly light-scattering fillers and the strongly light-scattering pigments are essential for the optical properties of the film layers, the first and the second film layer have different fillers and pigments.

The first film layer should either contain no fillers or those fillers whose refractive index is near to the refractive index of water. Examples of such fillers include, but are not limited to, silicone dioxide, silicates and aluminum silicates. A sodium aluminum silicate with the commercial name Transpafill® works well and has an average composition of 66% by weight SiO₂, 26% by weight Al₂O₃, 7% by weight Na₂O and 1% by weight SO₃. The average granulate size of particularly preferred primary particles is about 0.06 μm.

As noted above, the second layer should scatter light very strongly. The refractive index of the pigments in the second film layer should be at least 2.5. In some instances, titanium dioxide can be used. Particles with an average diameter of about 0.2 μm to about 0.8 μm have proven to be particularly advantageous. Easily processable titanium dioxide types in the anatase modification also can be used.

It is possible that the dry reagent composition is incorporated into one film layer, especially the first film layer. However, it is also possible that the dry reagent composition is incorporated in both film layers.

To optimize the test field in the carrier of the diagnostic test element, it has proven to be particularly advantageous when both film layers contain a non-hemolyzing wetting agent. Neutral (i.e., non-charged) wetting agents such as N-octanoyl-N-methyl glucamide can be used.

To produce the test field, the respective film layers each are produced successively from a homogeneous dispersion of the components. In this manner, the transparent foil is used as a base to form the coating compound for the first film layer. After the coating compound for the first film layer has been applied with a particular layer thickness, the layer is dried. Afterwards, the coating compound for the second layer is applied to this layer also with a thin layer thickness and subsequently dried. After drying, the thickness of the first film layer and the second film layer should be together no more than about 0.20 mm, no more than about 0.12 mm, or no more than about 0.08 mm.

The test field produced in this manner can be mounted on a supporting layer for better handling, those materials coming into consideration for such a layer that do not take up the liquid to be examined. Examples of such non-absorptive materials for the plastic foils include, but are not limited to, polystyrene, polyvinyl chloride, polyester, polycarbonate and polyamide. Likewise, metal foils or glass are suitable as further supporting materials.

In some embodiments, the detection side of the test field, which is to be observed and measured for a change in at least one optical property of the indicator reagent, should be visible through the supporting layer to determine the analyte to be detected in the body sample. This can be achieved by a transparent supporting layer. However, it is also possible that the supporting layer has a perforation that is covered by the detection side of the test field. The detection side then is visible through the perforation. In some instances, there can be a hole in the supporting layer below the detection side of the test field through which the detection side of the test field can be observed. The hole has a somewhat smaller diameter than the smallest linear dimension of the test field so that the test field outside the hole rests on the supporting layer and can be attached there.

Methods

Methods of the inventive concept can include methods of manufacturing a diagnostic test element. Such methods include at least a step of generating a dry reagent composition as described herein on a solid carrier.

Briefly, dry reagent compositions can be generated by dissolving the components first in a solvent or solvent mixture and subsequently removing the solvent or mixture of solvents by a suitable treatment such that the remaining composition is essentially free of the solvent or solvent mixture. Examples of treatments to remove the solvent or solvent mixture include, but are not limited to, heat treatment, evaporation, freeze drying and the like. In some instances, the treatment is heat treatment, especially heat treatment under the following conditions: heat treatment at about 60° C. or more for about 20 minutes to about 45 minutes or at about 95° C. for about 1 minute to about 2 minutes with heat circulation. Under these conditions, the dry reagent compositions can have a thickness of about 20 μm to about 200 μm or less; at a pressure of about 1 bar or about 0.1 bar. Moreover, it will be understood that to keep the composition under dry conditions, storage can be carried out in the presence of a drying agent. Examples of drying agents include, but are not limited to, silica gel, zeolites, calcium carbonate and magnesium sulfate.

As used herein, “solvent” means an agent that allows for dissolving the components of the dry reagent composition under conditions that do not irreversibly impair the function of the components of the composition and, in particular, the enzymatic activity of the dehydrogenase. Moreover, it is contemplated that the solvent dissolves the components under standard pressure (e.g., about 1 bar+/−10%) within a temperature range of about 5° C. to about 40° C. or at about 20° C.+/−10° C. Examples of solvents include, but are not limited to, water, water-based buffers such as phosphate buffered saline or Tris buffers, and alcohols such as hexanol, 2-methoxy-propanol, 2-methyl-2-butanol, citrate buffer, glycerine phosphate or Good's buffer (preferably in addition to Tris buffer). One of skill in the art understands that the solvent used can be a mixture of two or more of the aforementioned solvents. In some instances, solvent mixtures include mixtures of water or water-based buffers with alcohols.

In some instances, the generating step can include:
(i) applying a composition including components (a) to (e) of the dry reagent composition and a solvent (i.e., a composition including the components in a dissolved state rather than being dry) to a test field on the solid carrier; and
(ii) removing the solvent from the composition.

In other instances, the generating step can include:
(i) applying a first composition including components (a), (b), (d) and (e) of the dry reagent composition and a solvent (i.e., a composition including the components in a dissolved state rather than being dry) to a test field on the carrier in a first layer;
(ii) removing the solvent from the first layer;
(iii) applying a second composition including components (c) to (e) of the dry reagent composition and a solvent (i.e., a composition including the components in a dissolved state rather than being dry) in a second layer on the first layer; and
(iv) removing the solvent from the second layer.

In other instances, the generating step can include:
(i) applying a first composition including components (a), (b), (d) and (e) of the dry reagent composition and a solvent (i.e., a composition including the components in a dissolved state rather than being dry) to a test field on the carrier in a first layer;
(ii) removing the solvent from the first layer;
(iii) applying a second composition including components (b) to (e) of the dry reagent composition and a solvent (i.e., a composition including the components in a dissolved state rather than being dry) in a second layer on the first layer; and
(iv) removing the solvent from the second layer.

In other instances, the generating step includes:
(i) applying a first composition including components (a), (d) and (e) of the dry reagent composition and a solvent (i.e., a composition including the components in a dissolved state rather than being dry) to a test field on the carrier in a first layer;
(ii) removing the solvent from the first layer;
(iii) applying a second composition including components (b) to (e) of the dry reagent composition and a solvent (i.e., a composition including the components in a dissolved state rather than being dry) in a second layer on the first layer; and
(iv) removing the solvent from the second layer.

In other instances, the generating step includes:
(i) applying a first composition including components (c) to (e) of the dry reagent composition and a solvent (i.e., a composition including the components in a dissolved state rather than being dry) to a test field on the carrier in a first layer;
(ii) removing the solvent from the first layer;
(iii) applying a second composition including components (a), (b), (d) and (e) of the dry reagent composition and a solvent (i.e., a composition including the components in a dissolved state rather than being dry) in a second layer on the first layer; and
(iv) removing the solvent from the second layer.

In other instances, the generating step includes:
(i) applying a first composition including components (b) to (e) of the dry reagent composition and a solvent (i.e., a composition including the components in a dissolved state rather than being dry) to a test field on the carrier in a first layer;
(ii) removing the solvent from the first layer;
(iii) applying a second composition including components (a), (b), (d) and (e) of the dry reagent composition and a solvent (i.e., a composition including the components in a dissolved state rather than being dry) in a second layer on the first layer; and
(iv) removing the solvent from the second layer.

In other instances, the generating step includes:
(i) applying a first composition including components (b) to (e) of the dry reagent composition and a solvent (i.e., a composition including the components in a dissolved state rather than being dry) to a test field on the carrier in a first layer;
(ii) removing the solvent from the first layer;
(iii) applying a second composition including components (a), (d) and (e) of the dry reagent composition and a solvent (i.e., a composition including the components in a dissolved state rather than being dry) in a second layer on the first layer; and
(iv) removing the solvent from the second layer.

Typically, the dry reagent composition can be applied to a detection layer in the test field. The detection layer can be generated in particular by means of at least one wet chemical process (i.e., as one or more dispersions) such as aqueous dispersions of the dry reagent composition. Such layer-forming processes from one or more dispersions are known to one of skill in the art. See, e.g., EP Patent No. 0 821 234.

The solvent can be removed from the layers after application of the first and/or second composition to the test field of the test element by any techniques known for removing solvents including heat treatment, evaporation or freeze-drying. In some instances, the solvent is substantially removed by heat treatment.

Advantageously, the compatible solute reduces the decrease of the enzymatic activity of the at least one enzyme in the dry reagent composition and, in particular, during the substantial removal of the solvent and during the maintenance of the composition under dry conditions on the test element.

As part of the inventive concept, the generating steps incorporates using at least one compatible solute being ectoine or a derivative thereof for preventing a reduction of the enzymatic activity of at least one enzyme in a dry reagent composition under dry conditions, where the dry reagent composition comprises a dehydrogenase, a redox cofactor, an agent capable of eliciting a change in at least one optical property or an indicator reagent in the presence of redox equivalents, and an indicator reagent.

Methods of the inventive concept also can include methods of determining concentration or presence of an analyte in a body fluid sample with a test element as described herein. Such methods include steps of:
(a) contacting a diagnostic test element as described herein with a body fluid suspected of having an analyte of interest under conditions suitable for transforming at least one enzyme of a dry reagent chemistry to a reconstituted state; and
(b) measuring a change in at least one optical property of an indicator reagent in the wetted reagent composition including the at least one enzyme in the reconstituted state on the diagnostic test element, whereby the concentration or presence of the analyte of interest in the body fluid sample is determined.

As used herein “analyte” or “analyte of interest” means a biological molecule present in a body fluid sample the concentration or presence (or even absence) of which can be determined in accordance with the methods described herein. Since the determination is based upon enzymatic activity of a dehydrogenase, it is contemplated that the analyte is a substrate of the dehydrogenase included in the dry reagent compositions as described herein. Examples of analytes include, but are not limited to, glucose, maltose, mannose, galactose, glutamate, glucose-6-phosphate, ethanol and lactose.

As used herein, “body fluid sample” means any body fluid known or suspected of having the analyte to be determined. Examples of body fluids include, but are not limited to, blood including whole blood, plasma and serum, urine, saliva, liquor, synovial liquid and sudor.

As used herein, “contacting” means that the body fluid sample is applied to the solid carrier of the test element in a manner to allow for physical contact of the dry reagent composition of the test element by the solid carrier and the body fluid sample. In general, the contacting can be for a time and under conditions being sufficient to permit the dehydrogenase to be reconstituted (i.e., wetted and dissolved) and, thus, to become biologically active. Suitable conditions are known in the art. In some instances, the body fluid sample applied to the test element has a volume of less than about 2 μL or less than about 1 μL.

As used herein, “amount” or “concentration” means an absolute or relative amount or concentration of analyte present in a body fluid sample applied to the diagnostic test element. A relative amount is the concentration (i.e., the amount in relation to the volume).

As noted above, depending on the substrate specificity of the dehydrogenase to be used in the dry reagent composition on the diagnostic test element, different analytes can be determined by the methods.

Upon reconstitution of the biologically active dehydrogenase, the enzyme binds to its substrate (i.e., the analyte in the body fluid sample) and converts the analyte into the respective product and redox equivalents. The redox equivalents generated by the dehydrogenase allow for determining the dehydrogenase activity since the redox equivalents generated by the enzymatic conversion catalyzed by the dehydrogenase are transferred by the agent capable of eliciting a change in at least one optical property of the indicator reagent in the presence of redox equivalents. The change in the at least one optical property of the indicator reagent then can be measured.

Depending on the diagnostic test element, the measurement of the change of the optical property can be achieved by different techniques described elsewhere herein in more detail. For detecting the change of an optical property such as color, a spatially resolving optical detector may be used. As used herein, “spatially resolving optical detector” means an optical detector that has a multiplicity of optical sensors configured to record regions of the detection side of the detection layer that are not completely congruent. The spatially resolving optical detector can include at least one image sensor (i.e., an array of optical detectors) that can be one-dimensional or else two-dimensional. In some instances, the optical detector can be a CCD chip and/or CMOS chip.

The spatially resolving optical detector also can include at least one optical element for imaging the detection side and/or the detection layer onto an image-sensitive surface of the spatially resolving optical detector.

A change in at least one optical property measured by the methods described above shall be indicative for the presence of the analyte. One of skill in the art understands that to determine the amount/concentration of the analyte, it might be necessary to compare the extent of the change of the optical property. To this end, it might be necessary to compare a detected signal accompanying the optical change to signals accompanying optical changes elicited by known amounts of analytes (i.e., calibration signals). How such a calibration can be established is well known to one of skill in the art.

### EXAMPLES

The inventive concept will be more fully understood upon consideration of the following non-limiting examples, which are offered for purposes of illustration, not limitation.

### Example 1

### Generating Test Elements in the Form of Test Strips

Methods:

Four different reaction films for determining glucose levels were generated and coated on a foil essentially as described in EP Patent No. 0 821 234. The formulation of the first coat/film layer composition is shown below in Table 1.

TABLE 1
Components per 100 g for the First Layer (prior to drying)

| Component | Film 1 | Film 2 | Film 3 | Film 4 |
|---|---|---|---|---|
| GDH from B. subtilis | 1.09 g | 1.09 g | 1.09 g | 1.09 g |
| diaphorase from B. subtilis | 0.77 g | 0.77 g | 0.77 g | 0.77 g |
| NAD | 0.58 g | 0.58 g | 0.58 g | 0.58 g |
| Na/K phosphate buffer or HEPES | 0.35 g | 0.35 g | 0.35 g | 0.35 g |
| ectoine or hydroxyectoine | 0.00 g | 1.00 g | 2.00 g | 4.00 g |
| xanthan gum | 0.29 g | 0.29 g | 0.29 g | 0.29 g |
| silica FK 320DS | 5.80 g | 5.80 g | 5.80 g | 5.80 g |
| sodium-N-methyl-N-oleoyl-taurate | 0.03 g | 0.03 g | 0.03 g | 0.03 g |
| N-octanoyl-N-methyl-glucamide | 0.17 g | 0.17 g | 0.17 g | 0.17 g |
| polyvinylpyrrolidone | 0.86 g | 0.86 g | 0.86 g | 0.86 g |
| petraethylammoniumchloride | 0.07 g | 0.07 g | 0.07 g | 0.07 g |
| 2,18-phosphormolybdenic acid | 0.33 g | 0.33 g | 0.33 g | 0.33 g |
| hexasodium salt | | | | |
| polyvinylpropionate-dispersion (50 | 5.00 g | 5.00 g | 5.00 g | 5.00 g |
| Gew.-% in water) | | | | |
| K₃[Fe(CN)₆] | 0.01 g | 0.01 g | 0.01 g | 0.01 g |
| 2-methyl-2 butanol | 1.00 g | 1.00 g | 1.00 g | 1.00 g |
| add water up to 100 g | | | | |

The pH was adjusted to 6.8, and the composition was coated as a first film (about 120 μm) on a polycarbonate foil (125 μm). The coated composition was subsequently dried at 50° C.

A second coat was applied to the first coat on the foil. The formulation of the second coat/film layer composition is shown below in Table 2.

TABLE 2
Components of the Second Layer (prior to drying)

| Component | Film |
|---|---|
| Gantrez | 1.47 g |
| sodium-N-methyl-N-oleoyl-taurate | 0.03 g |
| PVP K25 | 2.01 g |
| Mega 8 | 0.37 g |
| tetraethylammoniumchloride | 0.45 g |
| silica FK 320DS | 2.00 g |
| titandioxide E171 | 22.00 g |
| polyvinylpropionate-dispersion (50 Gew.-% in water) | 6.25 g |
| bis-(2-hydroxyethyl)-(4-hydroximinocyclohexa-2,5- | 0.48 g |
| dienylidin)-ammoniumchloride | |
| 2,18-phosphormolybdenic acid hexasodium salt | 1.41 g |
| K₃[Fe(CN)₆] | 0.01 g |
| 2-methyl-2 butanol | 1.00 g |
| add water up to 100 g | |

The pH was adjusted to 6.8, and the composition was coated as a second film (about 25 μm) onto the first film coated on the foil. The coated composition was subsequently dried at 50° C. Test strips for glucose determination were generated as described in EP Patent No. 0 821 234.

### Example 2

### Determining Enzymatic Activity in Test Elements

Methods:

Test elements were stored in plastic vials in the presence of a drying agent for 6 weeks at 45° C. In a subsequent step, the test field of a test element was eluted by ultra-sonication using elution buffer. Enzymatic activity was determined in the supernatant.

TABLE 3
Elution Buffer and Detection Technique for the Different Enzymatic
Activities

| | Elution buffer | Detection technique |
|---|---|---|
| GDH | Tris/HCl, NaCl, NAD; | UV-detection at 340 nm |
| | pH 8.5 | |
| Diaphorase | Tris/HCl, NaCl, Triton; | INT → tetrazolium alsz; |
| | pH 8.8 | detected at 492 nm |

Results:

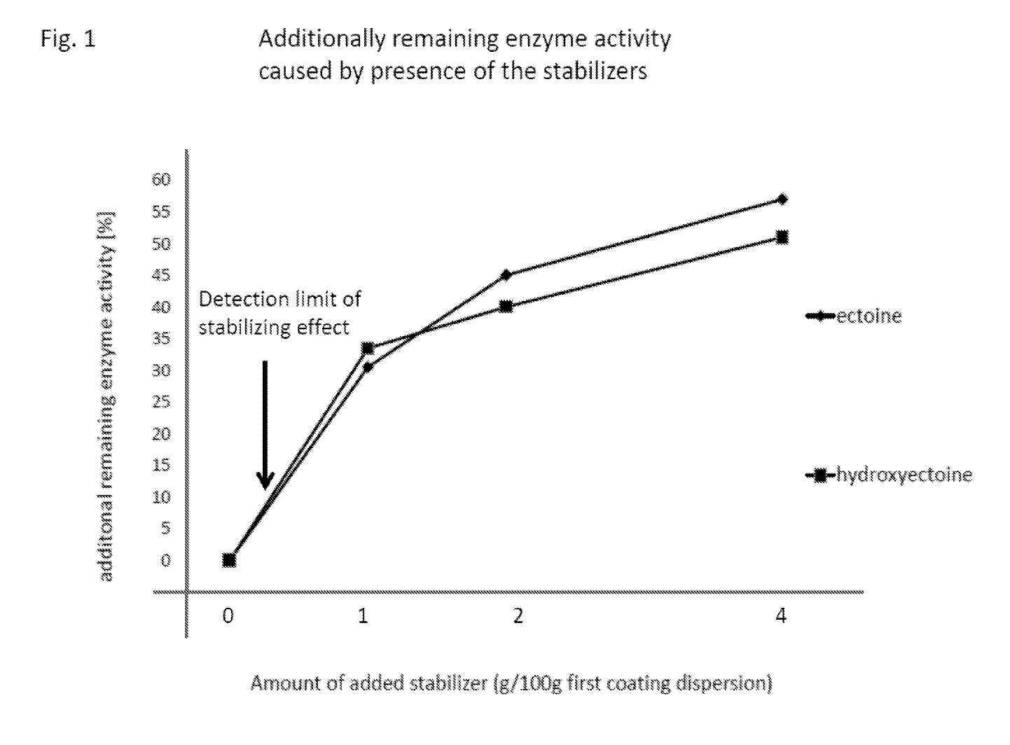

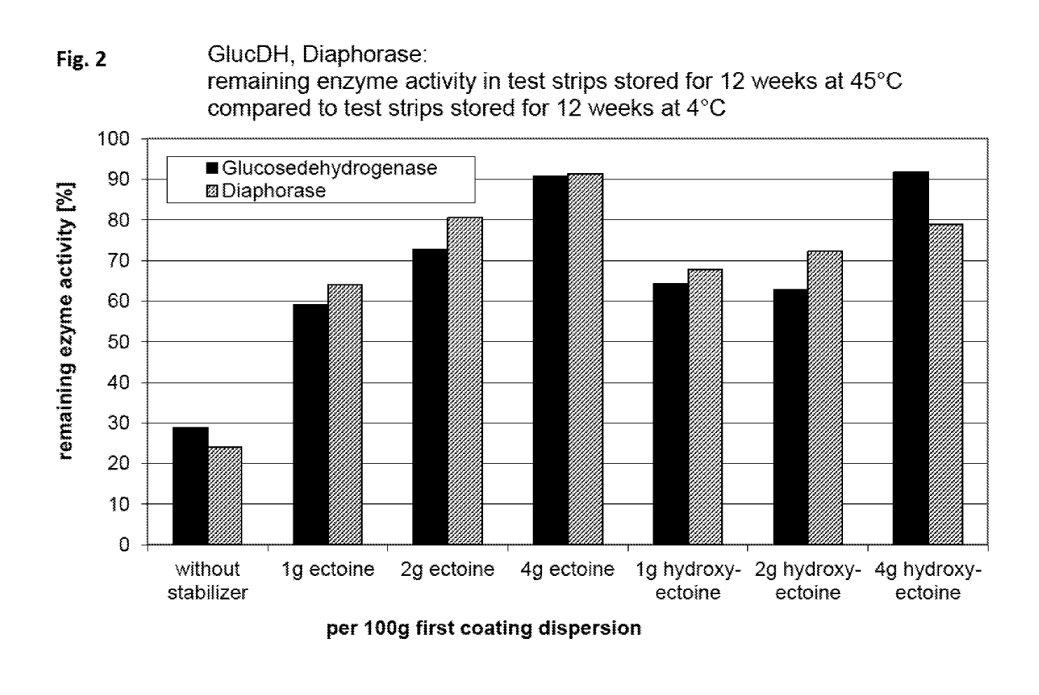

FIG. 1 shows that there is a significant stabilizing effect (more than 10%) for concentrations of ectoine of larger than 0.3 g/100 g coating composition. The same effect was observed for hydroxyectoine as shown in FIG. 2. Moreover, the figures show that the dehydrogenase, as well as the diaphorase, are stabilized.

### Example 3

### Determining Enzymatic Activity in Test Elements

Methods:

Test elements were stored in plastic vials in the presence of a drying agent for 63 days at 4° C. (KS), 24° C. (RT), 35° C. (DT) and 45° C. (HT). Test elements were used to determine blood glucose levels in a plurality of venous blood samples. The samples were measured with a reference method (Hitachi) in parallel. Results were normalized with respect to the KS stored test elements.

Results:

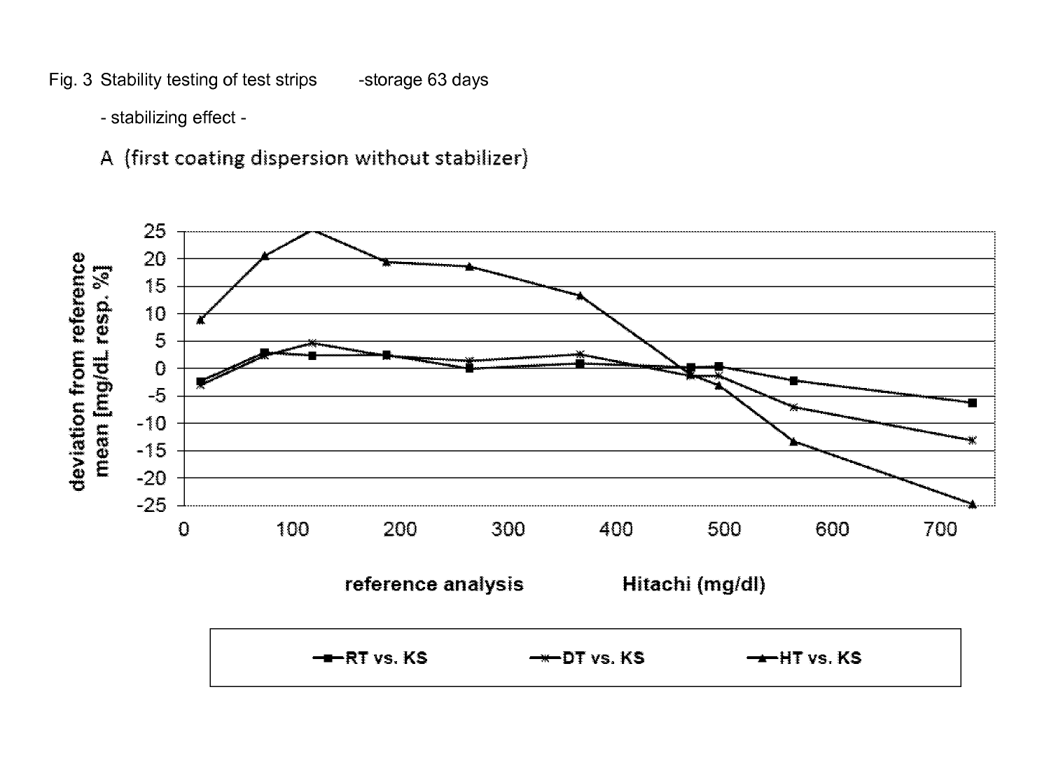

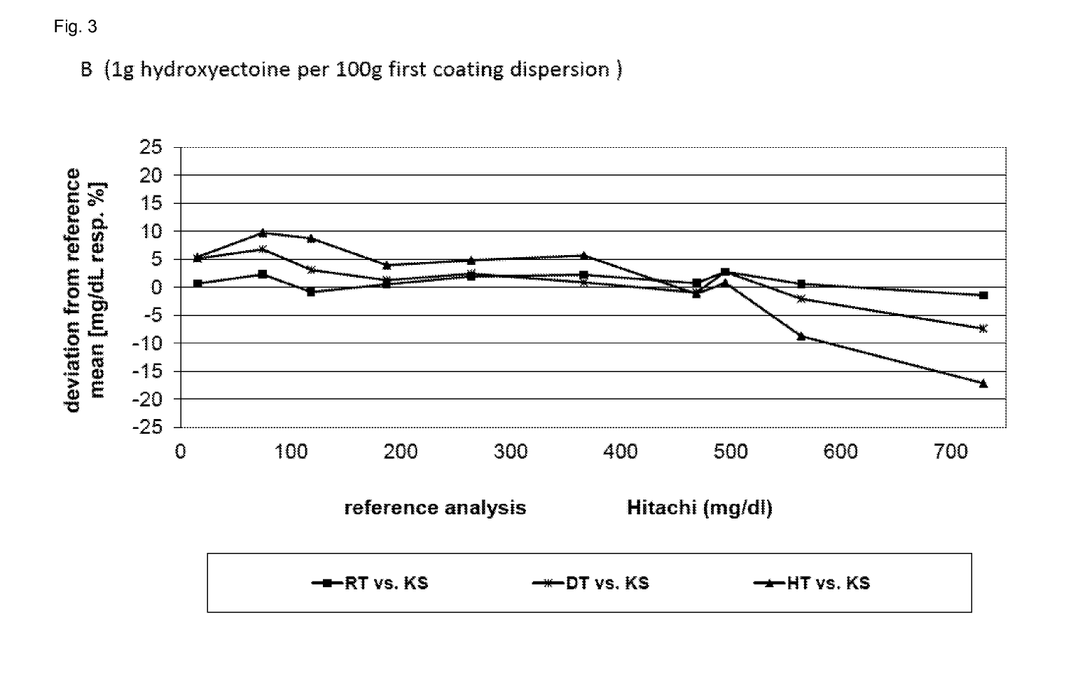

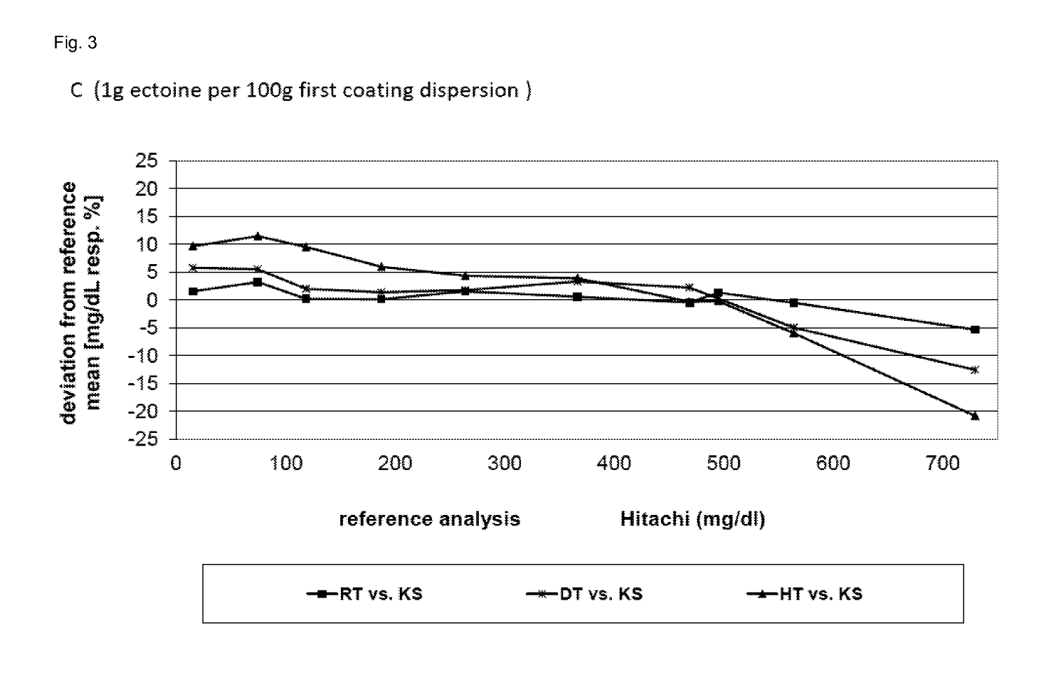

As shown in FIG. 3, the storage temperature is indicated as well as the measured glucose level as an indicator for enzymatic activity. As is evident, ectoine and hydroxyectoine act as stabilizers and preserve the enzymatic activities.

### Example 4

### Determining Enzymatic Activity in Test Elements with Different Buffers

Methods:

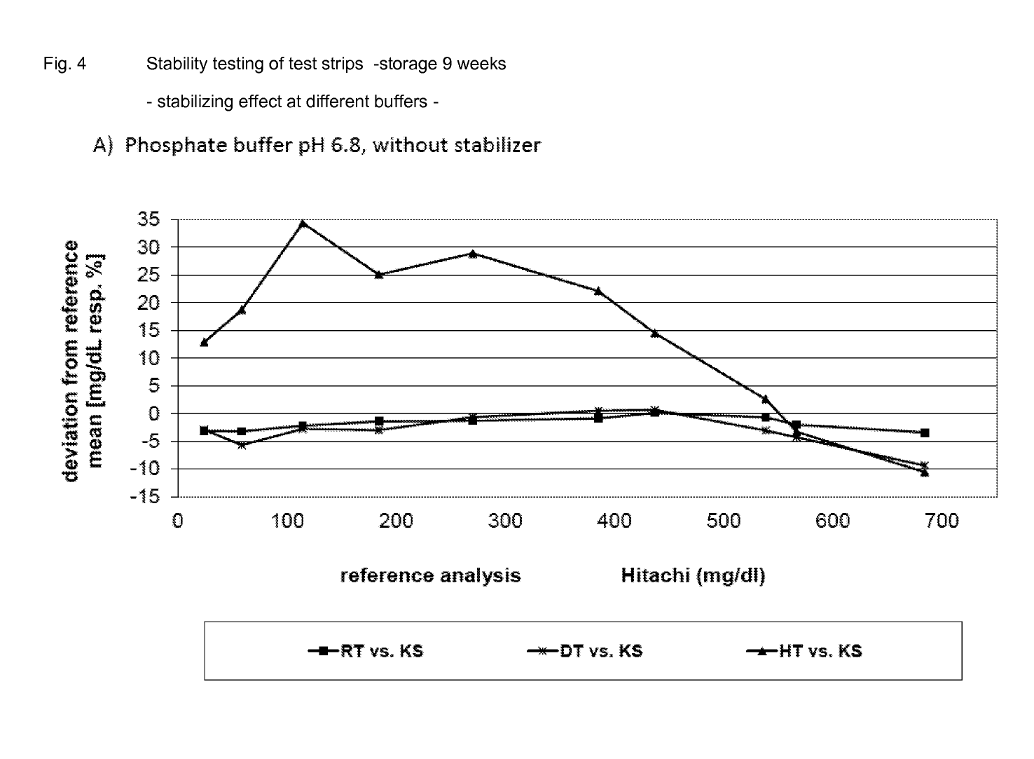

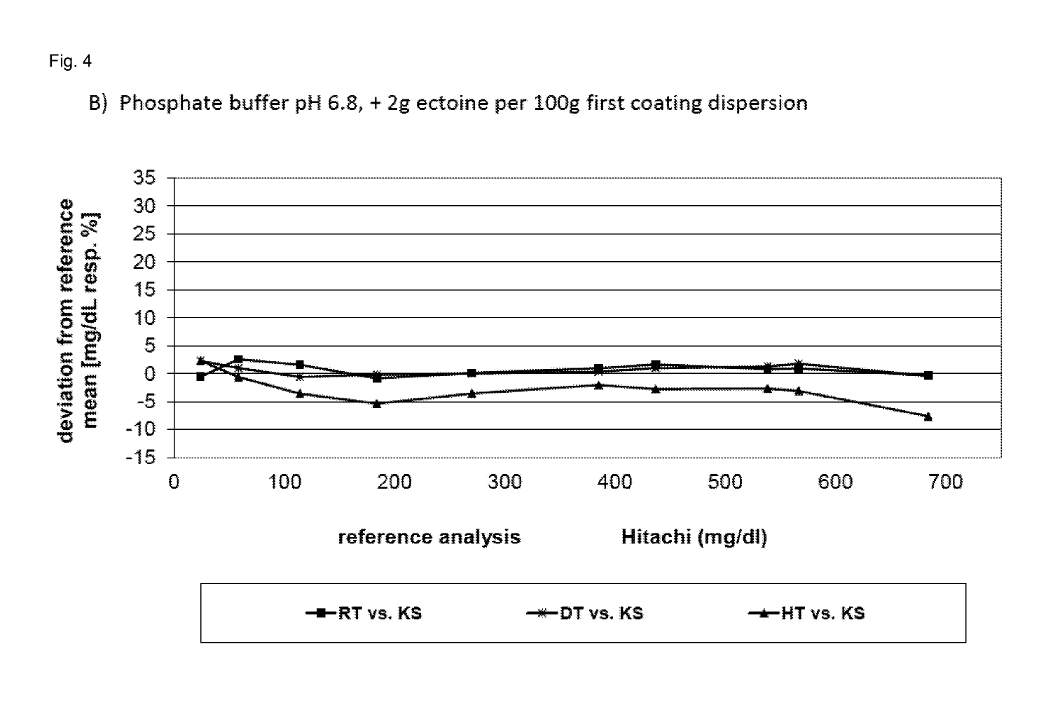

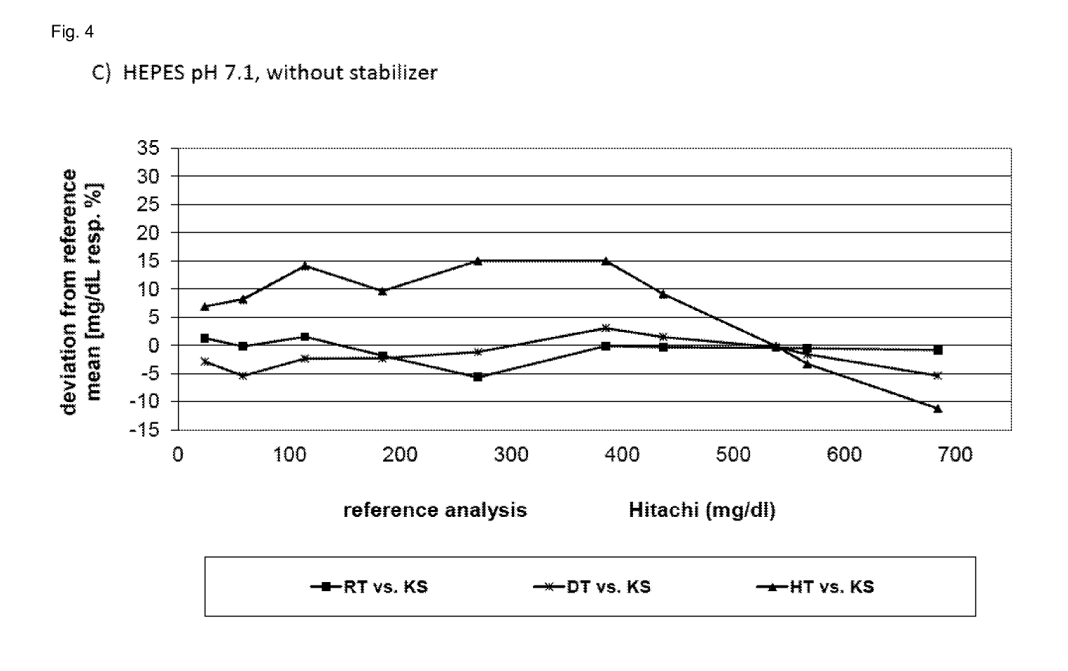

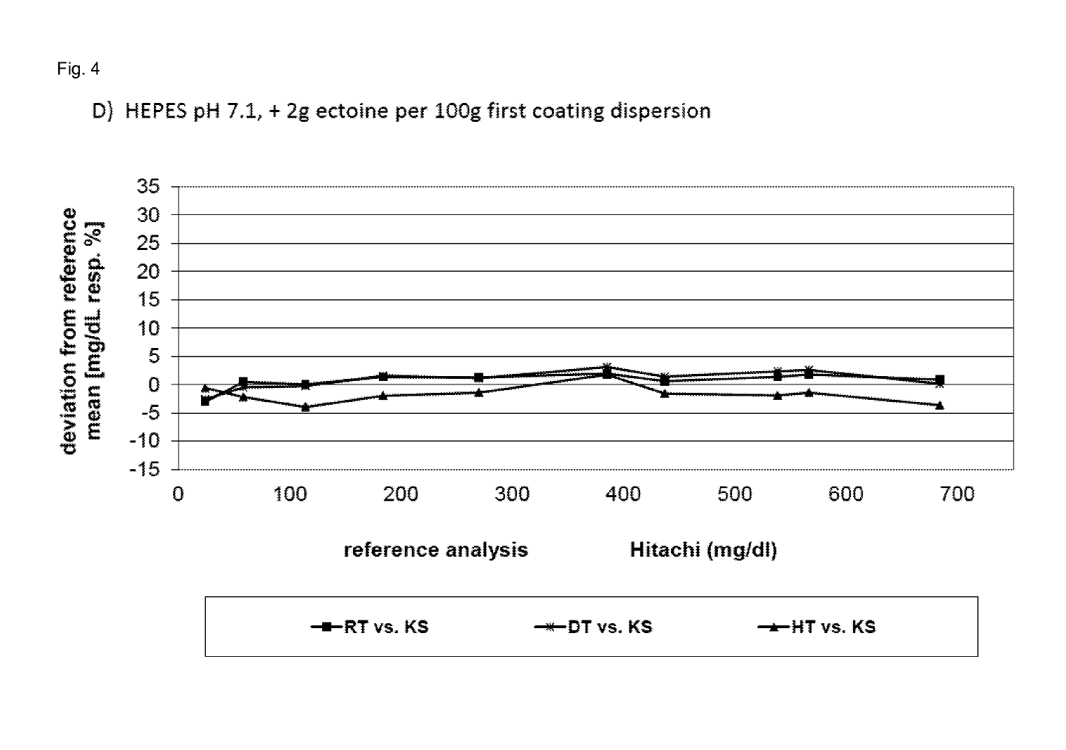

Test elements were stored and treated essentially as described in Example 3. Different buffers, namely a phosphate-buffer pH 6.8, without stabilizer, a phosphate-buffer pH 6.8, 2 g ectoine per 100 g first coating film, a HEPES buffer pH 7.1, without stabilizer, and a HEPES buffer pH 7.1, 2 g ectoine per 100 g first coating film, were used in the coating compositions as indicated in FIG. 4.

Results:

As shown in FIG. 4, the stabilizing activity of ectoine is observed independent of the buffer system.

### Example 5

### Determining Enzymatic Activity of a Glucose Dehydrogenase Mutant 2 in Solution and Dependency on Ectoine or Hydroxyectoine

Methods:

Aliquots of a solution including a glucose dehydrogenase mutant 2 as disclosed in WO Patent Application Publication No. 2011/020856 was combined with different amounts of ectoine or hydroxyectoine as indicated below in Table 4.

TABLE 4
Stabilizing Solutions

| Exp. | | |
|---|---|---|
| No. | Content | Stabilizer and Concentration |
| 1 | Glucose dehydrogenase Mut. 2, | 0 (reference) |
| | 3000 U/ml; NAD, 10 mg/ml; | |
| | K-Na phosphate, 15 mM, pH 6.8 | |
| 2 | dto | ectoine, 4% (w/v) |
| 3 | dto | ectoine, 2% (w/v) |
| 4 | dto | ectoine, 1% (w/v) |
| 5 | dto | hydroxyectoine, 4% (w/v) |
| 6 | dto | hydroxyectoine, 2% (w/v) |
| 7 | dto | hydroxyectoine, 1% (w/v) |

The aliquots were stored under different storage conditions (8 days, 4° C.; 8 days 35° C., 4 days 4° C. followed by 4 days 45° C.), and the enzymatic activity was determined after the storage.

Results:

The results are shown below in Table 5. No stabilizing effect of either ectoine or hydroxyectoine could be determined in solution.

TABLE 5
Enzymatic Activity

| Exp. | | | 8 days | 8 days | 4 days at 4° C., |
|---|---|---|---|---|---|
| No. | Note | Units | at 4° C. | at 35° C. | 4 days at 45° C. |
| 1 | reference | kU/g | 213 | 126 | 50 |
| 2 | ectoine, about 4% | kU/g | 207 | 137 | 57 |
| 3 | ectoine, about 2% | kU/g | 222 | 149 | 59 |
| 4 | ectoine, about 1% | kU/g | 222 | 144 | 60 |
| 5 | hydroxyectoine, | kU/g | 207 | 131 | 56 |
| | about 4% | | | | |
| 6 | hydroxyectoine, | kU/g | 221 | 141 | 60 |
| | about 2% | | | | |
| 7 | hydroxyectoine, | kU/g | 208 | 122 | 52 |
| | about 1% | | | | |

### Example 6

### Assessing Test Elements Including Gluc-DOR (a PQQ-Dependent GDH)

Methods:

A test element with Gluc-DOR and a mutant thereof (Gluc-DOR 31) was generated as described above in Example 1. The test element was analyzed as described above in Example 2. The determination of the enzymatic activity was carried out using nitrosoaniline as described in EP Patent No. 0 620 283.

Results:

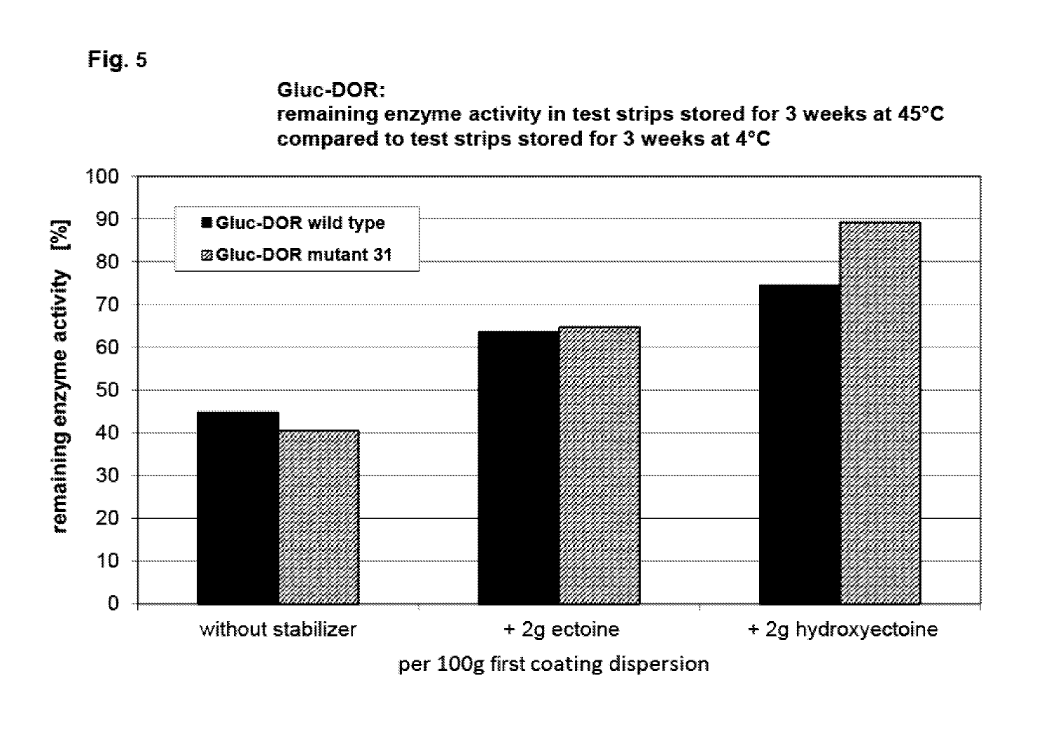

As shown in FIG. 5, a stabilizing effect was observable after 3 weeks at 45° C.

All of the patents, patent applications, patent application publications and other publications recited herein are hereby incorporated by reference as if set forth in their entirety.

The present inventive concept has been described in connection with what are presently considered to be the most practical and preferred embodiments. However, the inventive concept has been presented by way of illustration and is not intended to be limited to the disclosed embodiments. Accordingly, one of skill in the art will realize that the inventive concept is intended to encompass all modifications and alternative arrangements within the spirit and scope of the inventive concept as set forth in the appended claims.

The invention claimed is:

1. A dry composition comprising:
(a) a glucose dehydrogenase;
(b) a redox cofactor;
(c) an agent capable of eliciting a change in at least one optical property of an indicator reagent in the presence of redox equivalents;
(d) an indicator reagent; and
(e) at least one compatible solute being ectoine or a derivative thereof,
wherein the derivative of ectoine is selected from the group consisting of hydroxyectoine, homoectoine, a hydroxyectoine ester, a hydroxyectoine ether, a sulfonyl derivative of ectoine or an esterified sulfonyl derivative of ectoine and an amide of a sulfonyl derivative of ectoine.

2. The composition of claim 1, wherein the glucose dehydrogenase is selected from the group consisting of glucose dehydrogenase (EC number 1.1.1.47), glucose-6-phospate dehydrogenase (EC number 1.1.1.49), flavin adenine dinucleotide (FAD)-dependent glucose dehydrogenase (EC number 1.1.99.10), nicotinamide adenine dinucleotide (NAD)-dependent glucose dehydrogenase (EC number 1.1.1.119), pyrrolo quinoline quinine (PQQ)-dependent glucose dehydrogenase (EC number 1.1.5.2) and enzymatically active mutants thereof.

3. The composition of claim 1, wherein the agent capable of eliciting a change in the at least one optical property in the presence of redox equivalents transfers redox equivalents from the redox cofactor to the indicator reagent.

4. The composition of claim 3, wherein the agent capable of eliciting a change in the at least one optical property in the presence of redox equivalents is (i) a diaphorase selected from the group consisting of a lipoamide dehydrogenase and a NADH dehydrogenase, (ii) a phenazine selected from the group consisting of phenazinethosulfate, phenazinmethosulfate, 1-(3-carboxypropoxy)-5-ethylphenaziniumtrifluoro-methansulfonate and 1-methoxyphenazinmethosulfate, (iii) a nitrosoaniline, or (iv) a chinone selected from the group consisting of phenanthrenchinone, phenanthrolinchinone and benzo[h]-chinolinchinone.

5. The composition of claim 4, wherein the nitrosoaniline is [(4-nirosophenyl)imino]dimethanol-hydrochloride.

6. The composition of claim 1, wherein the redox cofactor is selected from the group consisting of carba-NAD, FAD, NAD and PQQ.

7. A diagnostic test element for determining an analyte concentration or presence in a body fluid sample, the test element comprising the dry reagent composition of claim 1 on a solid carrier.

8. The test element of claim 7, wherein the solid carrier comprises a test field containing the dry reagent composition, wherein the test field has a sample application side onto which the body fluid sample is applied and a detection side that allows for detecting a change in at least one optical property of the reagent when the analyte reacts with the dry reagent composition.

9. A method of manufacturing a diagnostic test element, the method comprising the step of generating a composition according to claim 1 on a solid carrier.

10. The method of claim 9, wherein the generating step comprises the steps of:
(i) applying a composition comprising components (a) to (e) of the dry reagent composition of claim 1 and a solvent to a test field on the carrier; and
(ii) removing the said solvent from the composition; or alternatively
(i) applying a first composition comprising components (a), (b), (d) and (e) of the dry reagent composition of claim 1 and a solvent to a test field on the carrier in a first layer;
(ii) removing the solvent from the first layer;
(iii) applying a second composition comprising components (c) to (e) of the dry reagent composition of claim 1 and a solvent in a second layer on the first layer; and
(iv) removing the solvent from the second layer; or alternatively
(i) applying a first composition comprising components (a), (b), (d) and (e) of the dry reagent composition of claim 1 and a solvent to a test field on the carrier in a first layer;
(ii) removing the solvent from the first layer;
(iii) applying a second composition comprising components (b) to (e) of the dry reagent composition of claim 1 and a solvent in a second layer on the first layer; and
(iv) removing the solvent from the second layer; or alternatively
(i) applying a first composition comprising components (a), (d) and (e) of the dry reagent composition of claim 1 and a solvent to a test field on the carrier in a first layer;
(ii) removing the solvent from the first layer;
(iii) applying a second composition comprising components (b) to (e) of the dry reagent composition of claim 1 and a solvent in a second layer on the first layer; and
(iv) removing the solvent from the second layer; or alternatively
(i) applying a first composition comprising components (c) to (e) of the dry reagent composition of claim 1 and a solvent to a test field on the carrier in a first layer;
(ii) removing the solvent from the first layer;
(iii) applying a second composition comprising components (a), (b), (d) and (e) of the dry reagent composition of claim 1 and a solvent in a second layer on the first layer; and
(iv) removing the solvent from the second layer; or alternatively
(i) applying a first composition comprising components (b) to (e) of the dry reagent composition of claim 1 and a solvent to a test field on the carrier in a first layer;
(ii) removing the solvent from the first layer;
(iii) applying a second composition comprising components (a), (b), (d) and (e) of the dry reagent composition of claim 1 and a solvent in a second layer on the first layer; and
(iv) removing the solvent from the second layer; or alternatively
(i) applying a first composition comprising components (b) to (e) of the dry reagent composition of claim 1 and a solvent to a test field on the carrier in a first layer;
(ii) removing the solvent from the first layer;
(iii) applying a second composition comprising components (a), (d) and (e) of the dry reagent composition of claim 1 and a solvent in a second layer on the first layer; and
(iv) removing the solvent from the second layer.

11. The method of claim 9, wherein the compatible solute reduces a decrease of enzymatic activity of at least one enzyme in the dry reagent composition under dry conditions.

12. The method of claim 9, wherein (a) is a glucose dehydrogenase or a mutant thereof, (b) is carba-NAD or NAD, (c) is a diaphorase or a nitrosoaniline, (d) is 2,18-phosphoromolybdenic acid, and (e) is ectoine or hydroxyectoine.

13. A method of determining amount or presence of an analyte in a body fluid sample, the method comprising the steps of:
(a) contacting the diagnostic test element of claim 7 with a body fluid suspected of having the analyte under conditions suitable for transforming the at least one enzyme to a reconstituted state in a wetted composition; and
(b) measuring a change in at least one optical property of the indicator reagent in the wetted composition comprising the at least one enzyme in the reconstituted state on the diagnostic test element, whereby the amount/concentration or presence of the analyte in the body fluid sample is determined.

14. The method of claim 13, wherein the analyte is glucose, and wherein (a) is a glucose dehydrogenase or a mutant thereof, (b) is carba-NAD or NAD, (c) is a diaphorase or a nitrosoaniline, (d) is 2,18-phosphoromolybdenic acid, and (e) is ectoine or hydroxyectoine.
